(19) United States
(12) Patent Application Publication
(10) Pub. No.: US 2019/0255014 A1
(43) Pub. Date: Aug. 22, 2019

(54) Therapeutic Cannabinoid Formulations and Methods for Their Use

(71) Applicant: Avidas Pharmaceuticals LLC, Doylestown, PA (US)

(72) Inventor: Margaret M. Gardner, Gladwyne, PA (US)

(73) Assignee: Avidas Pharmaceuticals LLC, Doylestown, PA (US)

(21) Appl. No.: 16/347,587

(22) PCT Filed: Nov. 6, 2017
(86) PCT No.: PCT/US2017/060248

§ 371 Date: May 5, 2019

Related U.S. Application Data
(60) Provisional application No. 62/418,756, filed on Nov. 7, 2016.

(51) Int. Cl. A61K 31/352; A61K 9/00; A61P 39/06

(52) U.S. Cl. CPC A61K 31/352; A61P 39/06; A61K 9/0014

(57) ABSTRACT

The present invention concerns formulations that provide for the transdermal delivery of canabinoid(s) in therapeutically effective amounts without the need for phospholipids or harsh irritating penetration enhancers while simultaneously delivering skin protecting/enhancing ingredients topically to the top of the skin for preventing and treating skin conditions and/or enhancing the appearance of skin.

### RELATED APPLICATION DATA

This PCT application claims priority to co-pending U.S. Provisional Patent Application No. 62/418,756, filed on Nov. 7, 2016, the entirety of which is hereby incorporated by reference.

### FIELD OF THE INVENTION

The present invention relates to compositions/formulations which contain therapeutically effective amounts of cannabinoids to be delivered into the skin of a mammal without the need for phospholipid(s) or harsh irritating penetration enhancers, while simultaneously delivering skin protecting/enhancing ingredients intended to remain substantially on the top of the skin for preventing, treating skin conditions and/or enhancing the appearance of skin. The formulations comprise cannabinoids in therapeutically effective amounts to improve the symptoms and side effects of disorders, diseases and the symptoms and side effects of their relative treatments including but not limited to auto-immune and auto-immune related disorders, cancer, osteoarthritic/muscoloskeletal disorders, psychological and neurological disorders, inflammation, fatigue, nausea, muscle and/or joint symptoms, pain, anxiety and paranoia, loss of appetite and seizures while simultaneously delivering skin protecting/enhancing ingredients to the top of the skin for preventing, treating skin conditions and/or enhancing the appearance skin.

### Background of Related Technology

Transdermal delivery methods are well known in the art but these deliveries often use penetration enhancers (Trommer, H., Neubert, R. H., Overcoming the stratum corneum: The modulation of Skin Penetration, A Review, Skin Pharmacol. Physiol. 2006; 19:106-121, DOI: 10.1159/000091978). Wallace teaches a topical CBD liniment delivery with solvent-based penetration enhancers (U.S. Pat. No. 6,949,582, Sep. 27, 2005), Stinchcomb teaches a transdermal CBD delivery for treating arthritis using diethylene glycol monoethyl ether as the penetration enhancer (U.S. Pat. No. 8,449,908, May 28, 2013) and Cristobal teaches a transcutaneous marijuana delivery using DMSO and glycols combined with heat. (U.S. Pat. No. 6,132,762). A common drawback of solvent-based penetration enhancers such as dimethyl sulfoxide (DSMO), alcohols and glycols is skin irritation (Paudel, K. S., et al., Challenges and Opportunities in Dermal Transdermal Delivery, Ther. Deliv. 2010, July; 1 (1): 109-131. PMCID: PMC2995530).

Phospholipids are also well-known permeation enhancers in the composition of vesicles, microemulsions and micellar systems including deliveries such as liposomes, lecithin organogels and transfersomes and require phospholipids such as phosphatidylcholine (PC), phosphatidylethanolamine (PE), phosphatidylserine (PS), phosphatidic acid (PA), phosphatidylinositol (PI), phosphatidylglycerol (PG) cardiolipin (CL), sphingomyelins (SM), or a mixture of various phospholipids to deliver ingredients transdermally. Smith teaches a CBD transdermal delivery requiring lecithin, a mixture of various phospholipids. (U.S. Pat. No. 9,375,417 Jun. 28, 2016). Many phospholipid-based transdermal deliveries contain lecithin, and soy lecithin is the most common type of lecithin. Commercial lecithin is predominately extracted from soy (aka soya). (https://www.ams.usda.gov/sites/default/files/media/Lecithin%20bleached%20TR%202009.pdf). Soy is one of the eight food allergens that fall under the labeling requirements of the Food Allergen Labeling and Consumer Protection Act (American College of Allergy, Asthma and Immunology, Soy Allergy, http://acaai.org/allergies/types/food-allergies/types-food-allergy/soy-allergy). The Arthritis Foundation reports that excess soy and other fatty acids can trigger the body to produce pro-inflammatory chemicals

Soy-based products produce potent estrogenic activity (Behr, M., et al, Estrogens in the daily diet: in vitro analysis indicates that estrogenic activity is omnipresent in foodstuff and infant formula, Food Chem. Toxicol. 2011 October; 49(10):2681-8. doi: 10.1016/j.fct.2011.07.039. Epub 2011 Jul. 23). Gynecomastia has been associated with soy consumption. Martinez, J., et al., “An unusual case of gynecomastia associated with soy product consumption,” Endocr. Pract., 2008 May-June; 14(4):415-8. Soy has a potentially adverse effect on development, Soy has been reported to stimulate estrogenic breast cancer. (Ju, Y. H., et al, Physiological concentrations of dietary genistein dose-dependently stimulate growth of estrogen-dependent human breast cancer (MCF-7) tumors implanted in athymic nude mice, J Nutr. 2001 November; 131(11): 2957-62). One in eight women are at risk for breast cancer and estrogen can cause hormone-receptor-positive breast cancers to develop and grow (http://www.breastcancer.org/risk/factors/hrt). Many doctors still recommend that women who take hormonal therapy or who have hormone receptor positive breast cancer avoid soy (http://www.breastcancer.org/tips/nutrition/reduce_risk/foods/soy). Soy has also been shown to reduce sperm count in men. (Chavarro, J. E., et al, “Soy food and isoflavone intake in relation to semen quality parameters among men from an infertility clinic.” Oxford Journals, Medicine & Health Human Reproduction, Volume 23, Issue 11 pp. 2584-590).

Topical deliveries of ingredients intended to protect and/or enhance the skin's appearance are intended to be delivered to the top of the skin and not transdermally. In fact, labeling of cosmetics and skin protecting ingredients advise that these ingredients should not be applied on broken skin, deep puncture wounds or serious burns. Their function is to largely to protect skin from damage such as external damage (wind, cold, UV radiation, etc), reactive oxygen species (peroxides, superoxides, oxidative stress), inflammation and intrinsic aging (cutaneous aging of the skin), to provide relief to conditions such as redness, rashes, chapping, chaffing, bites, cuts, scrapes, etc and improve the appearance of skin. Cosmetic ingredients to improve the condition and appearance of skin are well established and can be found in the Personal Care Products International Nomenclature of Cosmetic Ingredients. Examples of such cosmetic ingredients include emollients and conditioning agents. Examples of drug ingredients for skin protecting/enhancing include allantoin, aluminum hydroxide gel, calamine, cocoa butter, cod liver oil, vitamin A, cholecalciferol, colloidal oatmeal, dimethicone, glycerin, hard fat, kaolin, lanolin, mineral oil, petrolatum, sodium bicarbonate, topical starch, white petrolatum, zinc acetate, zinc carbonate, and zinc oxide. (Federal Register, Vol. 68, No. 107, Wednesday, Jun. 4, 2003 Rules and Regulations).

Skin protecting/enhancing ingredients are often combined with an external analgesic ingredient. (Federal Register, Vol. 68, No. 107, Wednesday, Jun. 4, 2003, Rules and Regulations). Sun protecting ingredients are also skin protecting ingredients and are often needed in topical formulations to prevent UV radiation, a carcinogen responsible for an estimated 5 million peopled treated for skin cancers each year and skin cancer deaths of nearly 9,000 people annually in the United States. (The Surgeon General's Call to Action to Prevent Skin Cancer, http://www.surgeongeneral.gov/library/calls/prevent-skin-cancer/call-to-action-prevent-skin-cancer.pdf) The American Academy of Dermatology reports that a lifetime cumulative UV damage to skin is also largely responsible for age-associated dryness and other cosmetic changes. Protecting the skin from the harmful effects of ultraviolet A (UVA) and B (UVB) radiation is therefore critical for reducing the risk of skin cancers and metastatic melanoma, and therefore recommends photoprotective measures be taken, including the use of sunscreen, whenever an individual is exposed to the sun. Again, these ingredients are intended to be delivered to the top of the skin to protect from UV radiation (absorb and reflect UV radiation).

### SUMMARY OF THE INVENTION

The present invention addresses certain problems and needs in the art by providing compositions that provide for the transdermal delivery of cannabinoids in therapeutically effective amounts without the need for penetration enhancers (e.g., solvents) or phospholipids while simultaneously delivering skin protecting/enhancing ingredients topically to the top of the skin for preventing, treating skin conditions and/or enhancing the appearance of skin. Such compositions according to the present invention include at least one cannabinoid compound present in a therapeutically effective amount; at least one skin protecting/enhancing ingredient; and a pharmaceutical carrier capable of delivering the cannabinoid transdermally while simultaneously delivering the skin protecting/enhancing agent topically (i.e., capable of bimodal delivery). Thus, the inventive compounds are useful for treating and improving symptoms and side effects of disorders and diseases including but not limited to auto-immune and auto-immune related disorders, cancer, osteoarthritic/muscoloskeletal disorders, inflammation, psychological and neurological disorders without the need for phospholipids or penetration enhancers while simultaneously delivering skin protecting/enhancing ingredients to the top of the skin to prevent, treat skin conditions and/or enhance the appearance of skin.

In certain non-limiting embodiments, the composition of the invention may include one or more cannabinoids, for example CBD and THC. In some embodiments the cannabinoid is cannabinol (CBN), cannabichromene (CBC) or their derivatives. In some embodiments the cannabinoid is tetrahydrocannabinol (THC), tetrahydrocannavivarin (THCV) or their derivatives. In some embodiments, the cannabinoid is cannabidiol (CBD) or cannabidiol acids or its derivatives. In some embodiments the cannabinoid is CBG, CBE, Iso-THC, CBL, CBT and other cannabinoids isolated from the Cannabis plant.

In certain non-limiting embodiments, the composition of the invention may include one or more cosmetic or drug ingredients to protect, treat skin conditions and/or enhance the skin, for example sun-protecting ingredients and dimethicone. In some embodiments the skin protecting/enhancing ingredients are sun-protecting ingredients. In some embodiments skin protecting/enhancing ingredients are allantoin, aluminum hydroxide gel, calamine, cocoa butter, cod liver oil, vitamin A, cholecalciferol, cannabidiol, colloidal oatmeal, dimethicone, emollients, glycerin, hard fat, kaolin, lanolin, mineral oil, vegetable oils, plant oils, petrolatum, skin conditioning agents, sodium bicarbonate, topical starch, white petrolatum, zinc acetate, zinc carbonate, and zinc oxide. Mixtures of two or more may also be used, for example zinc oxide and glycerin.

The instant invention includes methods for treating the symptoms and side effects of disorders, diseases and the symptoms and the side effects of their treatments including but not limited to auto-immune and auto-immune related disorders, cancer, osteoarthritic/muscoloskeletal disorders, psychological and neurological disorders, inflammation, fatigue, nausea, muscle and/or joint symptoms, pain, anxiety and paranoia, loss of appetite and seizures comprising administering a composition of the instant invention.

In certain non-limiting embodiments the invention is directed to topical formulations, including (a) a therapeutically effective amount of at least one cannabinoid compound; (b) at least one skin protecting/enhancing ingredient; and (c) a pharmaceutical carrier effective for simultaneous transdermal delivery of the at least one cannabinoid compound and topical delivery of the skin protecting/enhancing ingredient.

In certain embodiments, the pharmaceutical carrier does not include phospholipids.

In certain embodiments, the pharmaceutical carrier does not include lecithin or soy-based ingredients or harsh irritating penetration enhancers.

In certain embodiments, the at least one cannabinoid compound is selected from the group consisting of cannabigerol (CBG), cannabichromene (CBC), cannabidiol (CBD, CBDa and (−) trans-cannabidiol), cannabinol (CBN), tetrahydrocannabinol (THC), cannabicyclol (CBL), cannabielson (CBE), iso-tetrahydrocannabinol (iso-THC), cannabicitran (CBT), tetrahydrocannabivarin (THCV), decarboxilized tetrahydrocannabinol (THCa) and their various strains, cannabinoids, derivatives, metabolites, analogous, carbolic acid forms, cannabinoid acids, cannabinoid salts, CBDa, THCa, inactive forms, active forms, and combinations thereof.

In certain embodiments, the at least one skin protecting/enhancing ingredient is selected from the group consisting of allantoin, aluminum hydroxide gel, calamine, cocoa butter, cod liver oil, vitamin A, cholecalciferol, cannabidiol, colloidal oatmeal, dimethicone, glycerin, hard fat, kaolin, lanolin, mineral oil, petrolatum, vegetable oils, plant oils, silicones, sodium bicarbonate, topical starch, urea, petrolatum, zinc acetate, zinc carbonate, zinc oxide, sun-protecting ingredients, inactive forms, active forms, metabolites, and combinations thereof.

In certain embodiments, the pharmaceutically effective carrier is selected from the group consisting of water, amides, fatty acids, esters, pyrrolidones, surfactants, antioxidants, emulsifier/wetting agent, hydrocarbons, terpenes, urea, sterols, glycolipids, cyclodextrins and derivatives, combinations, and/or mixtures thereof.

In certain embodiments, the at least one antioxidant selected from the group consisting of ascorbic acid, ascorbyl palmitate, BHT, tocopheryl acetate, butylated hydroanisole (BHA), phenyl-anaphthylamine, hydroquinone, alpha-tocopherol, tocotrienol, cholecalciferol, propyl gallate, nordihydroquiaretic acid, niacinamide, arginine, Garcinia mangostana (Mangosteen) Peel Extract, Camellia sinensis (Green and White Tea) Leaf Extract, Punica granatum (Pomegranate) Extract, and derivatives, combinations, and mixtures thereof.

In certain embodiments, the at least one emulsifier/wetting agent is selected from the group consisting of mono and diglycerides and blends thereof, sorbitan fatty acid esters and blends thereof, fatty esters, fatty alcohols, mineral oils, polyether siloxane copolymers, polypropylene glycol (“PPG”)-15 stearyl ether, PPG-10 acetyl ether, PPG-4 lauryl ether, vitamin E acetate, PEG-7 glyceryl, polyoxyethylene sorbitan fatty acid ethers and blends thereof, polyoxyethylene sorbitol esthers, polyoxyethylene acids and blends thereof, polyoxyethylene acids and blends, polyoxyethylene alcohols and blends, polyoxyethylene adducts, ionic surfactants, calcium stearoyl lactylate, ceteareth-20, cetearyl glucoside, ceteth-10, ceteth-2, ceteth-20, cholesterol, cocamide MEA, glyceryl laurate, glyceryl stearate, glyceryl stearate and PEG-100 stearate, glyceryl stearate SE, glycol distearate, glycol stearate, isosteareth, lauramide DEA, laureth-23, laureth-4, linoleamide DEA, methyl glucose sesquistearate, oleth-8, oleth-10, oleth-10/polyoxyl 10 oleyl ether NF, oleth-2, oleth-20, PEG-100 stearate, PEG-20 almond glycerides, PEG-20 methyl glucose sesquistearate, PEG-25 hydrogenated castor oil, PEG-30 dipolyhydroxystearate, PEG-4 dilaurate, PEG-40 sorbitan peroleate, PEG-60 almond glycerides, PEG-8 laurate, PEG-80 sorbitan laurate, polysorbate 20, polysorbate 60, polysorbate 80, polysorbate 85, sodium stearoyl lactylate, sorbitan isostearate, sorbitan laurate, sorbitan oleate, sorbitan sesquioleate, sorbitan stearate, sorbitan stearate (and) sucrose cocoate, sorbitan trioleate, stearamide MEA, steareth-2, steareth-21 and derivatives, combinations and mixtures thereof.

In certain embodiments, the at least one cannabinoid compound, at least one skin protecting/enhancing ingredient, and pharmaceutical carrier are combined to form a cream, gel, liquid, lotion, solution, spray, emulsion, or a combination thereof.

In certain embodiments, the cannabinoid compound is about 0.01% to about 25% of the topical formulation.

In certain embodiments, the cannabinoid compound is about 0.05% to about 4%, about 0.1% to about 6%, about 0.4% to about 8%, about 0.5% to about 10%, about 1% to about 12%, about 1.1% to about 14%, about 1.5% to about 15%, about 2% to about 18%, about 2.5% to about 20%, about 3% to about 22%, about 3.5% to about 24%, about 4% to about 25%, or about 5% or more of the topical formulation by weight of the desired cannabinoid and the remaining ingredients are adjusted to correspond to the desired cannabinoid amount. If more than one cannabinoid is included or the cannabinoid potency is less than 98% purity, the cannabinoid compounds may be greater than 25% of the topical formulation.

In certain embodiments, the skin protecting/enhancing agent is about 0.01% to about 50% of the topical formulation.

In certain embodiments, the skin protecting/enhancing agent is about 1.0% to about 50%, about 2% to about 18%, about 2.5% to about 20%, about 3% to about 22%, about 3.5% to about 24%, about 4% to about 25%, about 5% to about 26%, about 6% to about 27%, about 7% to about 28%, about 8% to about 29%, about 9% to about 30%, about 10% to about 31%, about 11% to about 32%, about 12% to about 33%, abut 13% to about 34%, about 14% to about 35%, about 15% to about 36%, about 16% to about 37%, about 17% to about 38%, about 18% to about 39%, about 19% to about 40%, about 20% to about 41%, about 21% to about 42%, about 22% to about 43%, about 23% to about 44%, about 24% to about 45%, about 25% to about 46%, about 26% to about 47%, about 27% to about 48%, about 28% to about 49%, or about 29% to about 50% of the topical formulation.

In certain embodiments, the antioxidant is about 0.01% to about 20% of the topical formulation.

In certain embodiments, the antioxidant is 0<% to about 20%, 0<% to about 5%, about 1% to about 5%, about 2% to about 5%, about 3% to about 5%, about 4% to about 5%, about 5% to about 9%, about 5% to about 8%, about 5% to about 7%, about 5% to about 6%, about 3% to about 4%, about 3% to about 10%, about 3% to about 11%, about 3% to about 12%, about 3% to about 13%, about 3% to about 14%, about 3% to about 15%, about 3% to about 16%, about 3% to about 17%, about 3% to about 18%, about 3% to about 19%, about 3% to about 20%, or about 5% to about 20% of the topical formulation.

In certain embodiments, the emulsifier/wetting solution is about 1% to about 20% of the topical formulation.

In certain embodiments, the emulsifier/wetting solution is about 1% to about 20%, about 5% to about 20%, about 5% to about 15%, about 6% to about 14%, about 7% to about 13%, about 7% to about 12%, about 7% to about 11%, about 7% to about 10%, about 7% to about 9%, about 7% to about 8%, about 10% to about 13%, about 10% to about 12%, about 10% to about 11% or about 9% to about 11% of the topical formulation.

In certain embodiments, the aqueous component is about 35% to about 99% of the topical formulation.

In certain embodiments, the aqueous component is about 50% to about 95%, about 55% to about 95%, about 58% to about 95%, about 60% to about 95%, about 65% to about 95%, about 70% to about 95%, about 75% to about 95%, about 80% to about 95%, about 55% to about 60%, about 55% to about 65%, about 55% to about 70%, about 55% to about 75%, about 55% to about 80%, about 55% to about 85%, or about 65% to about 90% of the topical formulation.

In certain embodiments, the at least one cannabinoid compound is present in an amount therapeutically effective to improve the symptoms and side effects of disorders, diseases and their relative treatments' side effects and symptoms.

In certain embodiments, the skin protecting/enhancing agent is present in an amount effective to prevent, treat skin conditions and/or enhance the appearance of skin.

In certain embodiments, the skin condition is selected from the group consisting of external skin damage, skin damage resulting from reactive oxygen species, chemicals, drug therapies, radiation, skin irritants, inflammation, cutaneous skin aging, rashes, chapping, chaffing, bites, burns, cuts, and scrapes.

In certain non-limiting embodiments the invention is directed to topical formulations including (a) about 0.01% to about 25% of a therapeutically effective amount of at least one cannabinoid compound; and (e) about 0.01% to about 50% of at least one skin protecting/enhancing ingredient; and (c) a pharmaceutical carrier that does not require phospholipids or harsh irritating penetration enhancers, effective for simultaneous transdermal delivery of the at least one cannabinoid compound and topical delivery of the skin protecting/enhancing ingredient.

In certain embodiments, the pharmaceutical carrier includes a wetting agent/emulsifier and an antioxidant.

Finally, the instant invention includes methods for preventing, treating skin conditions and/or enhancing the appearance of skin such as the prevention of premature aging, skin cancer, dry skin, skin irritations such as rashes, chapping, chaffing, bites, cuts, scrapes, abrasions, erythema, minor burns, etc. comprising administering a composition of the instant inventions. The compositions in any embodiment of the present invention may be topically administered to a mammal in a single application, or may be topically administered to a mammal in multiple applications.

Various modifications and additions can be made to the embodiments without departing from the scope of the invention. Such modifications and additions are therefore considered to be part of this invention, without limitation imposed by the example embodiments described herein. Moreover, any word, term, phrase, feature, example, embodiment, or part or combination thereof, as used to describe or exemplify embodiments herein, unless unequivocally set forth as expressly uniquely defined or otherwise unequivocally.

### DETAILED DESCRIPTION OF THE INVENTION

Generally speaking, and as discussed in greater detail in the illustrative and non-limiting examples provided herein, the present invention is directed to cosmetic and pharmaceutical formulations/compositions (such terms being used interchangeably herein) that incorporate at least one therapeutically effective amount of cannabinoid that after application is capable of delivering the cannabinoid transdermally into the skin of a mammal without the need for phospholipids or harsh, irritating penetration enhancers to achieve a therapeutic effect.

In certain exemplary, non-limiting embodiments, the inventive cannabinoid compositions deliver cannabinoid transdermally in therapeutic amounts while simultaneously delivering skin protecting/enhancing ingredients which remain primarily on the top of the skin in order to protect, treat skin conditions and enhance the appearance of skin.

It is understood that the inventive formulations may be administered to any mammal in which they are effective in simultaneously delivering the composition comprising at least one cannabinoid delivered through the skin in a therapeutic amount combined with at least one skin protecting/enhancing ingredient to prevent, treat skin conditions and/or enhance the appearance of the skin of such mammal, and are particularly useful in mammals suited for transdermal drug delivery (such as humans, monkeys, pigs, and so forth). Therefore, the terms “mammal(s),” “individual(s),” and so forth as used herein are non-limiting and are to be construed broadly.

In certain exemplary, non-limiting embodiments, the inventive formulations include cannabinods in specific therapeutic amounts for treating mammals suffering from disorders, diseases and their symptoms and side effects including but not limited to auto-immune and auto-immune related disorders, cancer, osteoarthritic/muscoloskeletal disorders, psychological and neurological disorders, inflammation, fatigue, nausea, muscle and/or joint symptoms, pain, anxiety and paranoia, loss of appetite and seizures, while simultaneously preventing, treating skin conditions and/or improving the appearance of their skin.

In certain exemplary, non-limiting embodiments, the inventive formulations include cannabinods in specific therapeutic amounts for treating mammals suffering from the symptoms and side effects of treatments for disorders and diseases including but not limited to auto-immune and auto-immune related treatments, cancer and radiation treatments, osteoarthritic/muscoloskeletal treatments, inflammation, fatigue, nausea, muscle and/or joint symptoms, pain, anxiety and paranoia, loss of appetite and seizures, while simultaneously preventing, treating skin conditions and/or improving the appearance of their skin.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions and usages provided herein take precedent over any dictionary or extrinsic definition. That the present invention may be more readily understood, select terms are defined herein according to their usage.

As used herein, “cannabinoids” include, for example, phytocannabinoids found in Cannabis plants and other plants and synthetic cannabinoids.

As used herein, “cannabinoid(s)” refers, for example and without limitation, to any of known form of cannabinoid and mixtures. Examples of cannabinoids include cannabigerol (CBG), cannabichromene (CBC), cannabidiol (CBD, CBDa and (−) trans-cannabidiol), cannabinol (CBN), tetrahydrocannabinol (THC), cannabicyclol (CBL), cannabielson (CBE), iso-tetrahydrocannabinol (iso-THC), cannabicitran (CBT), tetrahydrocannabivarin (THCV), decarboxilized tetrahydrocannabinol (THCa) and their derivatives, metabolites and another analogous, and combinations thereof, as well as the various strains. For example, and without limitation carbolic acid forms of cannabinoids, cannabinoid acids, cannabinoid salts, CBDa or THCa.

As used herein, a “therapeutically effective amount” of a particular compound refers, for example and without limitation, to an amount of such compound that is effective to achieve a desired therapeutic result at a particular dosage, according to a particular dosing regimen, and over a particular period of time. The amount of a compound necessary to achieve a desired therapeutic result is influenced by, and will therefore vary based on, a number of factors, including for example and without limitation, the age, sex, and weight of the individual, factors that influence the metabolic rate of the individual, and any disorders and/or diseases of the individual (including the degree and severity thereof). Dosing regimens may be therefore be adjusted to achieve a desired therapeutic effect for a given individual. A “therapeutically effective amount” also refers to an amount at which negative factors, such as side effects and/or toxicity resulting from administration of the compound, are outweighed by the therapeutic benefits provided by administration of the compound. As used herein, for example and without limitation, a “therapeutically effective amount of Cannabinoid(s) in the inventive formulations refers to an amount of cannabinoid that is absorbed into the skin over a period of time to provide relief from the symptoms of diseases, disorders and/or their respective treatments. Examples and without limitation include relief from nausea and emesis and increases appetite in cancer and chemotherapy treatments, antispasm and anticonvulsion relief in neurological disorders, reduction in anxiety in auto-immune disorders and treatments, inhibition of tumor growth in cancers, reduce inflammation, pain relief and anti-spasmodic in osteoarthric and musculoskeletal disorders and treatments, reduced inflammation and antioxidant benefits in skin, and for treating overall symptoms of severe illnesses.

By way of further example and without limitation, a “therapeutically effective amount” of cannabinoid present in the inventive cannabinoid formulations is one in which improvement is realized in symptom or side effects with respect to one or more disorders, disease states and/or associated treatments in an mammal. Such symptoms and side effects include, for example and without limitation, symptoms and side effects of all known disease states, disorders and associated treatments regardless of whether environmental, genetic, lifestyle, physical activity, dietary and/or physiological factors.

By way of further example and without limitation, a “therapeutically effective amount” of cannabinoid present in the inventive formulations is one in which a specific amount of cannabinoid(s) is administered transdermally into the skin of an individual, in order to penetrate the skin for improve the appearance and health of the skin and/or transdermally to enter the blood stream. It is understood that those of ordinary skill in the art will, based on the teachings herein, be capable of empirically determining the therapeutically effective amount of cannabinoid needed in specific embodiments of the present inventive to achieve a particular therapeutic benefit, without the need for undue experimentation (as well as, in certain embodiments, determining therapeutically effective amounts of other agents that may be included in the inventive formulations in combination with cannabinoid, to provide various therapeutic benefits).

As the inventive formulations include skin protecting/enhancing ingredients, ingredients that prevent, treat skin conditions and/or enhance the appearance and are intended to remain mostly on the top of the skin, in various embodiments achieving a therapeutically effective amount of cannabinoid will take into account various factors attendant to the skin protecting/enhancing ingredients, for example and without limitation, that such formulations may be exposed to water (including alkaline salt water), may be partially removed by “towel drying” after a period of time, and so forth, and therefore in such embodiments such factors may be taken into account to ensure that a therapeutically effective amount of cannabinoid is administered to the individual, for example and without limitation, the concentration of cannabinoid, the delivery mechanism, and the inclusion of specific ingredients such as stabilizers, waterproofing agents, and so forth. In certain embodiments, it may be intended that the inventive formulations be pre-applied or re-applied after a certain period of time for particular therapeutic purposes, which will be taken into account in determining the concentration of cannabinoid and ingredients present in such formulations.

As the inventive formulations include one or more skin protecting/enhancing ingredients, exemplary ingredients include allantoin, aluminum hydroxide gel, calamine, cocoa butter, cod liver oil, vitamin A, cannabinoids, cholecalciferol, colloidal oatmeal, dimethicone, emollients, glycerin, hard fat, kaolin, lanolin, mineral oil, vegetable oils, plant oils, petrolatum, skin conditioning agents, sodium bicarbonate, topical starch, white petrolatum, zinc acetate, zinc carbonate, zinc oxide and derivatives, combinations, and mixtures thereof. In certain desired embodiments, the skin protecting/enhancing ingredients in the inventive formulation include sun protecting ingredients.

For example, the inventive formulations may include UV stabilizers.

For example, the inventive formulations may include UV radiation absorbers (sunscreen filters).

For example, the inventive formation may include sun protecting ingredients such as PABA, avobenzone, ecamsule, cinoxate, dioxybenzone, homosalate, menthyl anthranilate, octocrylene, octyl methoxycinnamate, octyl salicylate, oxybenzone, padimate, phenylbenzimidazole sulfonic, sulisobenzone, titanium dioxide, trolamine salicylate, zinc oxide, derivatives, combinations and mixtures thereof.

The inventive formulations may, in various exemplary, non-limiting embodiments, be provided in forms suitable for topical administration and that result in the transdermal delivery of a therapeutically effective amount of cannabinoid, for example and without limitation the inventive formulations may be provided as creams, gels, liquids, lotions, solutions, sprays, aerosols, and combinations thereof. In certain exemplary, non-limiting embodiments, the active agents, including cannabinoid, may be encapsulated (including microencapsulated) in the inventive formulations, for example, to be released when the encapsulation is ruptured under pressure, for time-release of the agent, and so forth. Suitable encapsulating materials and techniques, including those which release the encapsulated agent over time, are known in the art.

Other conventional cosmetic and/or pharmaceutical agents may be provided in the inventive formulations, so long as they are physiologically acceptable and suitable for use in combination with a therapeutically effective amount of cannabinoid in the formulation and skin protecting/enhancing ingredients.

For example, the inventive formulations may include physiologically compatible carriers and excipients, such as water, amides, fatty acids, esters, pyrrolidones, surfactants, antioxidants, emulsifiers/wetting agents, hydrocarbons, terpenes, urea, sterols, glycolipids, cyclodextrins and derivatives, combinations, and mixtures thereof. In certain desired embodiments, the inventive formulations include oil-free carriers.

For example, the inventive formulations may include emollients, such as fatty esters, fatty alcohols, mineral oils, polyether siloxane copolymers, polypropylene glycol (PPG)-stearyl ether, PPG-10 acetyl ether, steareth-10, oleth-8, PPG-4 lauryl ether, vitamin E acetate, PEG-7 glyceryl cocoate, lanolin, cholesterol, coconut oil, argan oil, cetyl alcohol, octyl hydroxystearate, dimethicone, cetyl alcohol, octyl hydroxystearate, dimethicone, and derivatives, combinations, and mixtures thereof.

For example, the inventive formulations may include skin conditioning agents, such as colloidal oatmeal, olive leaf, sulfonated shale oil, elubiol, 6-(1-piperidinyl)-2,4-pyrimidinediamine-3-oxide, finasteride, ketoconazole, zinc pyrithione, coal tar, benzoyl peroxide, selenium sulfide, hydrocortisone, pramoxine hydrochloride, tricetylammonium chloride, polyquaternium 10, panthenol, panthenol triacetate, vitamin B, vitamin C, vitamin D, vitamin E, vitamin K, keratin, lysine, arginine, hydrolyzed wheat proteins, hydrolyzed silk proteins, octyl methoxycinnamate, oxybenzone, minoxidil, titanium dioxide, zinc dioxide, erthromycin, tretinoin, octyl hydroxystearate; emollients, such as cholesterol NF, petrolatum, mineral oils and esters, including isopropyl myristate, isopropyl palmitate, 1-decene polymer (hydrogenated), and C12-C15 alcohol benzoates, and derivatives, combinations, and mixtures thereof.

For example, the inventive formulations may include pH stabilizing agent(s), such as butylated hydroxy toluene (BHT), ethylene diamine tetra acetic acid (EDTA), citric acid, triethanolamine (TEA), glycerin, propylene glycol, and derivatives, combinations, and mixtures thereof.

For example, the inventive formulations may include humectants, such as polyhydric alcohols, including glycerol/glycerin, polyalkylene glycols, alkylene polyols, including butylene glycol, propylene glycol, dipropylene glycol, polypropylene glycol, and polyethylene glycol, sorbitol, hydroxypropyl sorbitol, hexylene glycol, 1,3-dibutylene glycol, 1,2,6-hexanetriol, ethoxylated glycerol, propoxylated glycerol, and derivatives, combinations, and mixtures thereof.

For example, the inventive formulations may include buffering agents, such as citric acid, sodium citrate, and derivatives, combinations, and mixtures thereof.

For example, the inventive formulations may include viscosity adjusting agents, such as carbomer, gelling agents, zinc oxide, gum derivatives, and derivatives, combinations, and mixtures thereof.

For example, the inventive formulations may include preservatives, such as methylparaben, ethylparaben, butylparaben, propylparaben, phenoxyethanol, dmdm hydantoin, natural preservatives and derivatives, combinations, and mixtures thereof.

For example, the inventive formulations may include analgesics, such as aspirin, benzocaine, benzyl alcohol, butamban picrate, camphor, camphorated metacresol, cannabinoids, chloral hydrate, chlorobutanol, cyclomethycain sulfate, dibucaine, dibucaine hydrochloride, dimethisoquin hydrocholoride, diphenhydramine hydrochloride, dyclonine hydrochloride, eugenol, glycol salicylate, hexyiresorcinol, nydrocortisone, hydrocortisone acetate, junipar tar, lidocaine, lidocaine hydrochloride, menthol, methapyrilene hydrochloride, phenol, phenolate sodium, pramoxine hydrochloride, resorcinol, salicylamide, tetracaine, tetracaine hydrochloride, thymol, trolamine salicylate, tripiennamine hydrochloride, natural pain analgesics and derivatives, combinations and mixtures thereof.

For example, the inventive formulations may include wetting and emulsifying agents, such as polysorbate 20, polysorbitate 80, glyceryl distearate, POE 10 stearyl ether, steareth-2, steareth-4, steareth-6, steareth-7, steareth-10, steareth-11, steareth-13, steareth-15, St, steareth-4, steareth-20, ceateareth 20, stearyl alcohol, polyoxyethylene (2) stearyl or cetyl ether, ceteareth 20, cetearyl alcohol, and derivatives, combinations, and mixtures thereof. In certain desired embodiments, the inventive formulations include oil-free emulsifying agents.

For example, the inventive formulations may include chelating agents, such as ethylenediamine tetra acetic acid (EDTA), dihydroxyethyl glycine, tartaric acid, and derivatives, combinations, and mixtures thereof.

For example, the inventive formulations may include thickening agents, such as polyacrylamide, C13-C14 isoparaffin, laureth-7, and derivatives, combinations, and mixtures thereof.

For example, the inventive formulations may include antioxidants, such as ascorbic acid, ascorbyl palmitate, BHT, tocopheryl acetate, butylated hydroanisole (BHA), phenyl-anaphthylamine, hydroquinone, alpha-tocopherol, tocotrienol, cholecalciferol, propyl gallate, nordihydroquiaretic acid, niacinamide, arginine, Garcinia mangostana (Mangosteen) Peel Extract, Camellia sinensis (Green and White Tea) Leaf Extract, Punica granatum (Pomegranate) Extract, and derivatives, combinations, and mixtures thereof.

For example, the inventive formulations may include anti-irritant agents, such as allantoin, aloe, licorice extract, aloe, bisabolol, colloidal oatmeal, curcumin, petrolatum, ubiquinone and derivatives, combinations, and mixtures thereof.

For example, the inventive formulations may include fragrances, such as Eucalyptus oil, eucalyptol, camphor synthetic, peppermint oil, clove oil, lavender, chamomile, limonene, alpha pinene, beta pinene, myrcene, caryophyllene, linalool, citral, humulene, menthol, borneol, pulegone, sabinene, terpineol and thymol and derivatives, combinations, and mixtures thereof.

For example, the inventive formulations may include colorants.

The inventive formulations may also include mixtures and combinations and any of the above.

In certain embodiment the inventive formulation may have an SPF of about 2 to about 95, and in certain exemplary, non-limiting embodiments have an SPF of greater than 95. It is understood that the inventive formulations are not limited to any particular SPF or SPF range, and formulations having any SPF are contemplated in the present invention.

It is understood that one or more cannabinoids may be present in the inventive formulations in any suitable amount. For example, in certain exemplary, non-limiting embodiments, cannabinoid doses in the inventive formulations contain from about 0.1 to 100 milligrams of cannabinoid per dose and varies depending on the strain of cannabinoid and the purity and potency of the cannabinoid from about 0.2 to 25 mg, from about 0.4 to 50 mg, from about 2 to about 10 mg, from about 5 mg to about 20 mg and doses greater than 20 mg per dose.

It is understood that the amount of cannabinoid necessary to achieve a desired therapeutic result is influenced by, and will therefore vary based on, a number of factors, including for example and without limitation, the age, sex, and weight of the mammal, factors that influence the metabolic rate, and the specific disorders, diseases or related treatment symptoms of the mammal. The amount of at least one cannabinoid in the inventive formulation is between about 0.01% and about 25%. In one embodiment, the composition provides an individual dose of about 20 mg of cannabinoid.

Generally speaking, the bimodal pharmaceutical carrier described herein (i.e., capable of transdermal delivery of an active agent such as a cannabinoid compound and simultaneous topical delivery of an ingredient such as a skin protecting/enhancing ingredient) may be formed, for example, by combining a lipophilic phase (formed, for example, by mixing a wetting/emulsifier ingredients and an antioxidant with or without a non-phospholipid lipid such as a glycolipid or sterol) with an aqueous phase. The active agent (e.g., cannabinoid compound) to be delivered transdermally may be provided, for example, in the aqueous phase or may be otherwise added at this stage. Once the transdermal structures encapsulating the active agent are formed, the ingredient(s) to be delivered topically (e.g., skin protecting/enhancing ingredient(s)) may be added.

For example, the aqueous solution used to hydrate the lipophilic phase in forming the transdermal carrier, according to the present invention, may be in certain embodiments, a physiologically compatible solution such as water. The aqueous solution may have an active agent dissolved in it. The basic procedure according to this invention is to mix the wetting/emulsifier ingredients and antioxidant(s) (e.g., tocopherol, tocotrienol or mixtures thereof) to form a lipophilic phase. Non-phospholipid lipids or mixtures thereof (e.g. cholesterol) may be added to the lipophilic phase. Cannabinoid(s) are added and other transdermal actives may be added and mixed at this stage for incorporation into the transdermal carrier. Once the transdermal structures encapsulating the active ingredient are formed, skin protecting/enhancing ingredients, for example and without limitation topical active skin protecting/enhancing agent(s), are subsequently added and mixed along with other ingredients. Other ingredients added to the final composition may include additional cannabinoids and/or terpenes, and other ingredients such as preservatives, fragrances and viscosity adjusters.

Ingredient (is) intended to remain virtually on the top of the skin are added after the structures are formed, they are intentionally not enclosed in the transdermal carrier, and accordingly the topical formulations of the instant invention may, in such an embodiment, have a bimodal functionality, wherein the single cannabinoid formulation is capable of delivering cannabinoids transdermally without the need for phospholipids or harsh irritating penetration enhancers while concurrently delivering skin protecting/enhancing ingredients and other desired ingredients to the surface of the skin where they remain.

The transdermal delivery involves the application of the instant invention to the mammal's skin. A number of methods known in the art can be used to assess cannabinoids therapeutic effect. In one method, delivery may be assessed by measuring pain relief. Another method known in the art to assess cannabinoid transdermal delivery by wear testing and/or stamina effects testing. A well-known method of testing topical skin-protecting ingredients known in the art is sunscreen testing efficacy.

Further exemplary topical formulations of the present invention may be prepared by those of skill in the art, for example and without limitation, on the basis of the teachings provided herein, and according to the ranges of ingredients shown in Table 1.

TABLE 1
Exemplary Formulations

| Ingredient | Range of Amounts (w/w): |
|---|---|
| Cannabinoid | about .01% to about 25% |
| Cannabinoid | about .05% to about 4% |
| Cannabinoid | about 0.1% to about 6% |
| Cannabinoid | about 0.4% to about 8% |
| Cannabinoid | about 0.5% to about 10% |
| Cannabinoid | about 1% to about 12% |
| Cannabinoid | about 1.1% to about 14% |
| Cannabinoid | about 1.5% to about 15% |
| Cannabinoid | about 2% to about 18% |
| Cannabinoid | about 2.5% to about 20% |
| Cannabinoid | about 3% to about 22% |
| Cannabinoid | about 3.5% to about 24% |
| Cannabinoid | about 4% to about 25% |
| Cannabinoid | about 5% |
| Wetting Agent/Emulsifier | about 1% to about 20% |
| Wetting Agent/Emulsifier | about 5% to about 20% |
| Wetting Agent/Emulsifier | about 5% to about 15% |
| Wetting Agent/Emulsifier | about 6% to about 14% |
| Wetting Agent/Emulsifier | about 7% to about 13% |
| Wetting Agent/Emulsifier | about 7% to about 12% |
| Wetting Agent/Emulsifier | about 7% to about 11% |
| Wetting Agent/Emulsifier | about 7% to about 10% |
| Wetting Agent/Emulsifier | about 7% to about 9% |
| Wetting Agent/Emulsifier | about 7% to about 8% |
| Wetting Agent/Emulsifier | about 10% to about 13% |
| Wetting Agent/Emulsifier | about 10% to about 12% |
| Wetting Agent/Emulsifier | about 10% to about 11% |
| Wetting Agent/Emulsifier | about 9% to about 11% |
| Skin Protecting/Enhancing Ingredient | about 1.0% to about 50% |
| Skin Protecting/Enhancing Ingredient | about 2% to about 18% |
| Skin Protecting/Enhancing Ingredient | about 2.5% to about 20% |
| Skin Protecting/Enhancing Ingredient | about 3% to about 22% |
| Skin Protecting/Enhancing Ingredient | about 3.5% to about 24% |
| Skin Protecting/Enhancing Ingredient | about 4% to about 25% |
| Skin Protecting/Enhancing Ingredient | about 5% to about 26% |
| Skin Protecting/Enhancing Ingredient | about 6% to about 27% |
| Skin Protecting/Enhancing Ingredient | about 7% to about 28% |
| Skin Protecting/Enhancing Ingredient | about 8% to about 29% |
| Skin Protecting/Enhancing Ingredient | about 9% to about 30% |
| Skin Protecting/Enhancing Ingredient | about 10% to about 31% |
| Skin Protecting/Enhancing Ingredient | about 11% to about 32% |
| Skin Protecting/Enhancing Ingredient | about 12% to about 33% |
| Skin Protecting/Enhancing Ingredient | about 13% to about 34% |
| Skin Protecting/Enhancing Ingredient | abut 14% to about 35% |
| Skin Protecting/Enhancing Ingredient | about 15% to about 36% |
| Skin Protecting/Enhancing Ingredient | about 16% to about 37% |
| Skin Protecting/Enhancing Ingredient | about 17% to about 38% |
| Skin Protecting/Enhancing Ingredient | about 18% to about 39% |
| Skin Protecting/Enhancing Ingredient | about 19% to about 40% |
| Skin Protecting/Enhancing Ingredient | about 20% to about 41% |
| Skin Protecting/Enhancing Ingredient | about 21% to about 42% |
| Skin Protecting/Enhancing Ingredient | about 22% to about 43% |
| Skin Protecting/Enhancing Ingredient | about 23% to about 44% |
| Skin Protecting/Enhancing Ingredient | about 24% to about 45% |
| Skin Protecting/Enhancing Ingredient | about 25% to about 46% |
| Skin Protecting/Enhancing Ingredient | about 26% to about 47% |
| Skin Protecting/Enhancing Ingredient | about 27% to about 48% |
| Skin Protecting/Enhancing Ingredient | about 28% to about 49% |
| Skin Protecting/Enhancing Ingredient | about 29% to about 50% |
| Aqueous Component | about 35% to about 99% |
| Aqueous Component | about 50% to about 95% |
| Aqueous Component | about 55% to about 95% |
| Aqueous Component | about 58% to about 95% |
| Aqueous Component | about 60% to about 95% |
| Aqueous Component | about 65% to about 95% |
| Aqueous Component | about 70% to about 95% |
| Aqueous Component | about 75% to about 95% |
| Aqueous Component | about 80% to about 95% |
| Aqueous Component | about 55% to about 60% |
| Aqueous Component | about 55% to about 65% |
| Aqueous Component | about 55% to about 70% |
| Aqueous Component | about 55% to about 75% |
| Aqueous Component | about 55% to about 80% |
| Aqueous Component | about 55% to about 85% |
| Aqueous Component | about 65% to about 90% |
| Antioxidant or Mixture | 0 < % to about 20% |
| Antioxidant or Mixture | 0 < % to about 5% |
| Antioxidant or Mixture | about 1% to about 5% |
| Antioxidant or Mixture | about 2% to about 5% |
| Antioxidant or Mixture | about 3% to about 5% |
| Antioxidant or Mixture | about 4% to about 5% |
| Antioxidant or Mixture | about 5% to about 9% |
| Antioxidant or Mixture | about 5% to about 8% |
| Antioxidant or Mixture | about 5% to about 7% |
| Antioxidant or Mixture | about 5% to about 6% |
| Antioxidant or Mixture | about 3% to about 4% |
| Antioxidant or Mixture | about 3% to about 10% |
| Antioxidant or Mixture | about 3% to about 11% |
| Antioxidant or Mixture | about 3% to about 12% |
| Antioxidant or Mixture | about 3% to about 13% |
| Antioxidant or Mixture | about 3% to about 14% |
| Antioxidant or Mixture | about 3% to about 15% |
| Antioxidant or Mixture | about 3% to about 16% |
| Antioxidant or Mixture | about 3% to about 17% |
| Antioxidant or Mixture | about 3% to about 18% |
| Antioxidant or Mixture | about 3% to about 19% |
| Antioxidant or Mixture | about 3% to about 20% |
| Antioxidant or Mixture | about 5% to about 20% |

If more than one cannabinoid is included or the cannabinoid potency is less than 98% purity, the cannabinoid compounds may be greater than 25% of the topical formulation.

One of skill in the art will understand that the ingredients in the final formulation must total 100% and, based on the teachings provided herein, will understand that modifications to the exemplary formulations provided herein are possible (e.g., replacement of a recited ingredient with a different ingredient, addition of a different ingredient, and/or modification of an amount of an ingredient) provided that such modifications result in a bimodal formulation as taught and described herein (i.e., capable of delivering an active agent such as a cannabinoid transdermally while simultaneously delivering a skin protecting/enhancing agent topically). One example, as described herein, is use of a non-phospholipid lipid or mixture in place of, or incorporated as part of, or in addition to, the antioxidant or antioxidant mixture, to form the lipophilic phase.

The discussion herein and the following Examples set forth and illustrate various exemplary embodiments of the present invention, which are understood to be illustrative and non-limiting.

### Example 1: Skin Protecting/Enhancing Cannabinoid Formulation for Improving Stamina (Energy & Strength)

A cannabinoid formulation without the need for phospholipids or harsh irritating penetration enhancers is prepared according to present invention to have at least one cannabinoid compound present in a therapeutically effective amount; at least one skin protecting/enhancing agent; and a bimodal pharmaceutical carrier. The formulation is useful for improving stamina effects (energy & strength).

The therapeutically effective cannabinoid improves energetic effects concurrently while the skin protecting/enhancing agent(s) which, when applied topically, permits the more energetic mammal to engage in outdoor activity while reducing or eliminating skin damage resulting from environmental exposure (damage from UVA and/or UVB rays, and/or wind and/or cold, etc). This reduced skin damage is beneficial to mammals with autoimmune diseases or disorders and/or mammals being treated for auto-immune diseases or disorders where the disease or disorder and/or treatments result in fatigue and lack of energy as well as photosensitive and compromised skin, and thus these inventive formulations are provided with concurrent transdermal administration of cannabinoid(s) and topical administration of skin protecting/enhancing agent(s) to compensate for the multiple symptoms and side effects associated with the disease or disorder.

Various formulations according to this Example may be provided in which the specific concentrations of cannabinoid, skin protecting/enhancing agent(s), and other ingredients, are selected for each formulation based on the specific intended use of the resulting formulation, including the environmental and other conditions in which the formulation is intended to be used, whether the formulation is intended to be re-applied after particular activities and/or after specific periods of time, and specific characteristics of the mammal that may impact the transdermal delivery of cannabinoid in that mammal.

Accordingly, these inventive formulations may be provided for use by a number of mammals engaged in varied activities and using these inventive formulations under varied conditions, while in all cases simultaneously delivering cannabinoid transdermally while topically preventing, treating skin conditions and/or enhancing the skin in such mammals.

### Example 2: Skin Protecting/Enhancing Cannabinoid Formulation Providing Systemic and Topical Pain Relief

A cannabinoid formulation capable of transdermal delivery without the need for phospholipids or harsh irritating penetration enhancers is prepared according to the present invention to have at least one cannabinoid compound present in a therapeutically effective amount; at least one skin protecting/enhancing agent; and a bimodal pharmaceutical carrier for improving the symptoms and side effects of cancer and cancer treatments.

In such formulations, the choice of cannabinoid agent(s) will depend on a number of factors, including the age, weight, severity of side effects and symptoms, including, for example symptoms and side effects of cancer and/or cancer treatments, (including, for example pain and chemotherapy and/or radiation induced skin irritation). In all cases, the inventive formulation provides a therapeutically effective amount cannabinoid that is administered transdermally to relieve pain, while providing skin protection/enhancement to compromised skin caused by the disorder, disease and/or caused by the relevant treatment for the disorder. As with the inventive formulations discussed in Example 1, these inventive treatment formulations may be provided with specific concentration of cannabinoid combined with skin protecting/enhancing ingredients, and other ingredients, selected for each inventive formulation based on the specific intended use of the resulting inventive formulation, including the environmental and other conditions in which the inventive formulation is intended to be used, and whether the inventive formulation is intended to be re-applied before or after particular activities and/or after specific periods of time, and also taking into account specific characteristics of the mammal for whom it is intended that may impact cannabinoid delivery in such mammal.

Accordingly, these inventive treatment formulations may be provided for use by a number of mammals engaged in varied activities and using these inventive treatment formulations under varied conditions, while in all cases delivering therapeutic amounts of cannabinoid for pain while providing skin protection/enhancement such as relief for skin irritations and/or improved appearance of skin caused by harmful effects of radiation, environmental, chemicals, drug therapies and other skin irritants.

### Example 3: Skin Protecting/Enhancing Cannabinoid Formulations for Treating the Symptoms of Disorders, Diseases and their Relevant Treatments for Severe Chronic Diseases

A cannabinoid formulation without the need for phospholipids or harsh irritating penetration enhancers is prepared according to the present invention to have at least one cannabinoid compound present in a therapeutically effective amount; at least one skin protecting/enhancing agent; and a bimodal pharmaceutical carrier for improving the symptoms and side effects of severe chronic diseases.

As discussed herein, such disorders and/or diseases which may be treated using the inventive formulations include, for example and without limitation, disorders and diseases and their relative treatments associated with autoimmune disorders and diseases, auto-immune and auto-immune related disorders, cancer, osteoarthritic/muscoloskeletal disorders, infectious disease sequella, psychological and neurological disorders, inflammation, fatigue, nausea, muscle and/or joint symptoms, pain, anxiety and paranoia, loss of appetite, spasms and seizures.

As with the formulations discussed above, in these inventive treatment formulations, the choice of cannabinoid and skin protecting/enhancing agent(s) will depend on a number of factors, including the desired symptom relief of the resulting inventive formulation. For example, a higher amount of cannabinoid may be desired by individuals with pain compared to individuals with a loss of appetite; a higher amount of skin protecting/enhancing ingredients may be desired by individuals who desire skin protecting/enhancing ingredients that do not minimize or eliminate, UVA and/or UVB exposure as the area of the body for the product application will not be exposed to UVA/UVB radiation. For example osteoarthritic joint pain may require a cannabinoid like CBD that has shown efficacy data in treating inflammation and arthritis. As the product may be applied directly to the joint to benefit from a local effect, the skin protecting/enhancing ingredients may be zinc oxide or allantoin for their anti-irritant benefits.

In all cases, the inventive formulation provides a therapeutically effective amount cannabinoid that is administered transdermally to relieve pain, while providing relief to skin irritation caused by the disorder, disease and/or relevant treatment.

As with the inventive formulations discussed in Example 1, these inventive treatment formulations may be provided with specific concentration of cannabinoid combined with skin protecting/enhancing ingredients, and other ingredients, selected for each inventive formulation based on the specific intended use of the resulting inventive formulation, including the environmental and other conditions in which the inventive formulation is intended to be used, and whether the inventive formulation is intended to be pre-applied or re-applied after particular activities and/or after specific periods of time, and also taking into account specific characteristics of the mammal for whom it is intended that may impact cannabinoid delivery in such mammal.

Accordingly, these inventive treatment formulations may be provided for use by a number of mammals engaged in varied activities and using these inventive treatment formulations under varied conditions, while in all cases delivering therapeutic amounts of cannabinoid for pain while providing skin protection/enhancement such as relief for skin irritations and/or enhanced appearance of skin caused by harmful effects of radiation, environmental, chemicals, drug therapies and other skin irritants.

### Example 4: Formulations

From the teachings provided herein, those of skill in the art will be able to make the inventive formulations having one or more skin protecting/enhancing agents and a therapeutically effective amount of cannabinoid, and test the efficacy of such inventive formulations in established animal models (for example and without limitation, animal models suited for topical and transdermal drug delivery) and using conventional pharmacokinetic analysis and techniques, as well as prepare such inventive formulations using ingredients to render them suitable for use by particular mammals, for use during particular activities, and/or for use when exposed to particular external conditions.

Animal models that may be useful for testing the efficacy of the inventive compounds are known in the art, see for example Soares, et al. “Evaluation of the role of the cannabidiol system in an animal model of ischemia/reperfusion kidney injury.” Rev. Bras. Ter. Intensiva. 2015 October-December; 27(4):383-9; Xiong, et al. “Cannabinoids suppress inflammatory and neuropathic pain by targeting α3 glycine receptors.” J. Exp. Med. 2012 Jun. 4; 209(6):1121-34 (reporting cannabinoids suppress inflammatory and neuropathic pain); Shoval, et al. “Prohedonic Effect of Cannabidiol in a Rat Model of Depression.” Neuropsychobiology 2016; 73(2):123-9 (reporting oral cannabidiol in rat models for depression, suggesting that CBD may be an effective and safe anxiolytic); Giacoppo, et al. “A new formulation of cannabidiol in cream shows therapeutic effects in a mouse model of experimental autoimmune encephalomyelitis.” Daru 2015 Oct. 21; 23:48 (reporting CBD solubilized in propylene glycol in mice may exert neuroprotective effects against autoimmune encephalomyelitis (EAE); Indorato, et al. “The therapeutic use of cannabinoids: Forensic aspects.” Forensic Sci. Int. 2016 August; 265:200-3 (reporting inhaled (not smoked) cannabinoids and oral mucosal cannabinoids have been used for medical treatments including Multiple Sclerosis (MS) in humans); Zettl, et al. “Evidence for the efficacy and effectiveness of THC-CBD oromucosal spray in symptom management of patients with spasticity due to multiple sclerosis.” Ther. Adv. Neurol. Disord. 2016 January; 9(1):9-30 (reporting THC-CBD efficacy and effectiveness for spacitity with oromucosal spray in humans); Reznik, et al. “Cannabidiol: a potential treatment for post Ebola syndrome?” Int. J. Infect. Dis. 2016 Sep. 26; 52:74-76 (reporting oral CBD has been used to treat fatigue, confusion, memory, anxiety, depression and anorexia post Ebolla sequelae in humans); Wilsey, et al. “An Exploratory Human Laboratory Experiment Evaluating Vaporized Cannabis in the Treatment of Neuropathic Pain From Spinal Cord Injury and Disease.” J. Pain 2016 September; 17(9):982-1000 (reporting effect of vaporized Cannabis on neuropathic pain in humans); and Giacoppo, et al. “Purified Cannabidiol, the main non-psychotropic component of Cannabis sativa, alone, counteracts neuronal apoptosis in experimental multiple sclerosis.” Eur. Rev. Med. Pharmacol. Sci. 2015 December; 19(24):4906-19 (reporting the effects of intraperitoneal administration of CBD and demonstrated antiapoptotic power against the neurodegenerative processes underlying MS in mice).

It is understood that skin protecting/enhancing sunscreen formulations are conventionally tested at a skin concentration of about 2 mg/cm² and therefore it is contemplated that the formulations with sunscreens as the skin protecting/enhancing agent(s) may be tested at or about such concentration.

A lipohilic composition of 28 grams (14% w/w) of wetting/emulsifying ingredient combined with antioxidants are mixed. The aqueous solution is used to hydrate the lipophilic phase. 6 grams (3% w/w) of CBD are added to the mixture. A composition of skin protecting/enhancing ingredients 10 grams (5% w/w) are added to the aqueous and lipophilic mixture and mixed. Once thoroughly mixed, preservatives (1% w/w), antioxidants and viscosity adjusters (2% w/w) are added.

A composition of 25.4 grams of wetting/emulsifying ingredient and antioxidants are mixed. The aqueous solution is used to hydrate the lipophilic phase. 6.6 grams of THC-a are added to the mixture. A composition of skin protecting/enhancing ingredients 16 grams are added and mixed. Preservatives and other excipients are added.

A composition of 33.0 grams of wetting/emulsifying ingredient and antioxidant are mixed. The aqueous solution is used to hydrate the lipophilic phase. 8 grams of CBD and 2 grams of THC are added to the mixture. A composition of skin protecting/enhancing ingredients 30 grams are added and mixed. Preservatives, terpenes and other excipients are added.

### Example 5: Study Results

Research and development was conducted in a cohort of human subjects (4) to assess therapeutic delivery of the cannabidiol formulation and its efficacy to improve pain and stamina. The formulation included the active ingredient cannabidiol and active skin protecting/enhancing ingredients in a carrier to deliver the cannabidiol transdermally and the skin protecting/enhancing ingredients topically. The inclusion criteria for the study was subjects suffering from mild, moderate or severe pain. The subjects were asked to identify the site of their pain and indicate their baseline pain using the universal pain assessment tool (Scale of 1-10). Subjects applied one gram of the cannabidiol (CBD) formulation to the site of the pain or asked to apply to the wrists for overall pain. Pain was assessed at 10 minutes, 20 minutes, 30 minutes, 2 hours, 4 hours, six hours and 12 hours following one gram product application. Baseline pain was a mean of 5.5 (moderate). After ten minutes, mean pain was reduced (−9%), pain relief continued after 20 minutes (mean −27%) and was substantially reduced after 30 minutes (−40%) and was sustained for 4 hours (−40%) following product application.

A study was conducted in a cohort of adult subjects (4) to assess stamina (energy and strength) following one gram of product application. Stamina was assessed using a five-point ordinal sale: 0=no change, 1=minimal, 2=mild, 3=moderate, 4=meaningful). The subjects were required to report their findings after 10 minutes, 20 minutes, 30 minutes and 2 hours following product application. There was virtually no change after 10 minutes, after 20 minutes 50% of the subjects had mild improvements in stamina and 75% of the subjects had mild-moderate improvements in stamina after 30 minutes. Subjects reported improvements in stamina lasted though the 2 hour timeframe in all subjects but one.

A study was conducted in subjects (n=4) to assess wear testing and skin irritation using an ordinal scale (0=none, 1=minimal, 2=some, 3=moderate, 4=measurable) following one gram product application after 10 minutes, 20 minutes, 30 minutes and 60 minutes. There was virtually no product residual after 10 minutes and no skin irritation was reported. The study formulation contained a cannabidiol as the transdermal ingredient and the skin-protecting/enhancing topical ingredients were glycerin and dimethicone, two well researched anti-irritants. The results of the study demonstrated fast absorption with anti-irritant skin protecting/enhancing benefits to the skin

Once given the above disclosure, many other features, modifications, and improvements will become apparent to the skilled artisan. Such features, modifications, and improvements are therefore considered to be part of this invention, without limitation imposed by the example embodiments described herein. Moreover, any word, term, phrase, feature, example, embodiment, or part or combination thereof, as used to describe or exemplify embodiments herein, unless unequivocally set forth as expressly uniquely defined or otherwise unequivocally set forth as limiting, is not intended to impart a narrowing scope to the invention in contravention of the ordinary meaning of the claim terms by which the scope of the patent property rights shall otherwise be determined. All references discussed and disclosed herein are hereby incorporated by reference in their entirety.

### REFERENCES

Chavarro, et al. “Soy food and isoflavone intake in relation to semen quality parameters among men from an infertility clinic.” Oxford Journals, Medicine & Health Human Reproduction. Volume 23, Issue 11 pp. 2584-590.
Federal Register, Vol. 68, No. 107, Wednesday, Jun. 4, 2003 Rules and Regulations
Food Chem. Toxicol. 2011 October; 49(10):2681-8 (doi: 10.1016/j.fct.2011.07.039; Epub 2011 Jul. 23).
Giacoppo, et al. “Purified Cannabidiol, the main non-psychotropic component of Cannabis sativa, alone, counteracts neuronal apoptosis in experimental multiple sclerosis.” Eur. Rev. Med. Pharmacol. Sci. 2015 December; 19(24):4906-19.
Giacoppo, et al. “A new formulation of cannabidiol in cream shows therapeutic effects in a mouse model of experimental autoimmune encephalomyelitis.” Daru 2015 Oct. 21; 23:48 (doi: 10.1186/s40199-015-0131-8)
Gorter, R. W. “Cancer cachexia and cannabinoids.” Forsch Komplementarmed. 1999 October; 6 Suppl 3:21-2.
Indorato, et al. “The therapeutic use of cannabinoids: Forensic aspects.” Forensic Sci. Int. 2016 August; 265:200-3 (doi: 10.1016/j.forsciint.2016.03.031; Epub 2016 Mar. 23).
Ju, et al. “Physiological concentrations of dietary genistein dose-dependently stimulate growth of estrogen-dependent human breast cancer (MCF-7) tumors implanted in athymic nude mice.” J. Nutr. 2001 November; 131(11): 2957-62.
Paudel, et al. “Challenges and Opportunities in Dermal Transdermal Delivery” Ther. Deliv. 2010, July; 1 (1): 109-131.
Reznik, et al. “Cannabidiol: a potential treatment for post Ebola syndrome?” Int. J. Infect. Dis. 2016 Sep. 26; 52:74-76 (doi: 10.1016/j.ijid.2016.09.020; Epub ahead of print]
Santos, et al. “The neuroprotection of cannabidiol against MPP⁺-induced toxicity in PC12 cells involves trkA receptors, upregulation of axonal and synaptic proteins, neuritogenesis, and might be relevant to Parkinson's disease.” Toxicol In Vitro. 2015 Dec. 25; 30(1 Pt B):231-40. doi: 10.1016/j.tiv.2015.11.004. Epub 2015 Nov. 7.
Soares, et al. “Evaluation of the role of the cannabidiol system in an animal model of ischemia/reperfusion kidney injury.” Rev Bras Ter Intensiva. 2015 October-December; 27(4):383-9. doi: 10.5935/0103-507X.20150064.
Soares and Campos, “Evidences for the anti-panic actions of Cannabidiol.” Curr. Neuropharmacol. 2016 May 9. [Epub ahead of print]
Shoval, et al. “Prohedonic Effect of Cannabidiol in a Rat Model of Depression.” Neuropsychobiology 2016; 73(2):123-9 (doi: 10.1159/000443890; Epub 2016 Mar. 25).
Trommer and Neubert. “Overcoming the stratum corneum: The modulation of Skin Penetration, A Review” Skin Phamacol. Physiol. 2006; 19:106-121 (DOI: 10.1159/000091978).
Wilsey, et al. “An Exploratory Human Laboratory Experiment Evaluating Vaporized Cannabis in the Treatment of Neuropathic Pain From Spinal Cord Injury and Disease.” J. Pain 2016 September; 17(9):982-1000 (doi: 10.1016/j.jpain.2016.05.010; Epub 2016 Jun. 7).
Xiong, et al. “Cannabinoids suppress inflammatory and neuropathic pain by targeting α3 glycine receptors.” J. Exp. Med. 2012 Jun. 4; 209(6):1121-34 (doi: 10.1084/jem.20120242; Epub 2012 May 14).
Zettl, et al. “Evidence for the efficacy and effectiveness of THC-CBD oromucosal spray in symptom management of patients with spasticity due to multiple sclerosis.” Ther. Adv. Neurol. Disord. 2016 January; 9(1):9-30 (doi: 10.1177/1756285615612659).
http://www.surgeongeneral.gov/library/call s/prevent-skin-cancer/call-to-action-prevent-skin-cancer.pdf
https://www.ams.usda.gov/sites/default/files/media/Lecithin%20bleached%20TR%202009.pdf
http://acaai.org/allergies/types/food-allergies/types-food-allergy/soy-allergy
http://www.breastcancer.org/risk/factors/hrt
http://www.breastcancer.org/tips/nutrition/reduce_risk/foods/soy
U.S. Pat. No. 6,113,940
U.S. Pat. No. 6,132,762
U.S. Pat. No. 6,328,992
U.S. Pat. No. 6,949,582
U.S. Pat. No. 8,449,908
U.S. Pat. No. 9,095,555
U.S. Pat. No. 9,095,563
U.S. Pat. No. 9,168,278
U.S. Pat. No. 9,186,386
U.S. Pat. No. 9,205,063
U.S. Pat. No. 9,375,417
U.S. Pat. No. 9,380,813
United States Patent Publication No. 2011/0052694
United States Patent Publication No. 2014/0302148

What is claimed is:

1. A topical formulation, comprising: (a) a therapeutically effective amount of at least one cannabinoid compound; (b) at least one skin protecting/enhancing ingredient; and (c) a pharmaceutical carrier effective for simultaneous transdermal delivery of said at least one cannabinoid compound and topical delivery of said skin protecting/enhancing ingredient.

2. A topical formulation according to claim 1, wherein said pharmaceutical carrier does not include phospholipids.

3. A topical formulation according to claim 1, wherein said pharmaceutical carrier does not include lecithin, soy, or soy-based ingredients.

4. A topical formulation according to claim 1, wherein said pharmaceutical carrier does not include a penetration enhancer.

5. A topical formulation according to claim 1, wherein said at least one cannabinoid compound is selected from the group consisting of cannabigerol (CBG), cannabichromene (CBC), cannabidiol (CBD, CBDa and (−) trans-cannabidiol), cannabinol (CBN), tetrahydrocannabinol (THC), cannabicyclol (CBL), cannabielson (CBE), iso-tetrahydrocannabinol (iso-THC), cannabicitran (CBT), tetrahydrocannabivarin (THCV), decarboxilized tetrahydrocannabinol (THCa) and their various strains, cannabinoids, derivatives, metabolites, analogous, carbolic acid forms, cannabinoid acids, cannabinoid salts, CBDa, THCa, inactive forms, active forms, and combinations thereof.

6. A topical formulation according to claim 1, wherein said at least one skin protecting/enhancing ingredient is selected from the group consisting of allantoin, aluminum hydroxide gel, calamine, cocoa butter, cod liver oil, vitamin A, cannabidiol, cholecalciferol, colloidal oatmeal, dimethicone, glycerin, hard fat, kaolin, lanolin, mineral oil, petrolatum, vegetable oils, plant oils, silicones, sodium bicarbonate, topical starch, urea, petrolatum, zinc acetate, zinc carbonate, zinc oxide, sun-protecting ingredients, inactive forms, active forms, metabolites, and combinations thereof.

7. A topical formulation according to claim 1, wherein said pharmaceutically effective carrier comprises of water, amides, fatty acids, esters, pyrrolidones, surfactants, one or more antioxidants, emulsifier/wetting agent, hydrocarbons, terpenes, urea, sterols, glycolipids, cyclodextrins and derivatives, combinations, and/or mixtures thereof.

8. A topical formulation according to claim 6, wherein said one or more antioxidants is selected from the group consisting of ascorbic acid, ascorbyl palmitate, BHT, tocopheryl acetate, butylated hydroanisole (BHA), phenyl-anaphthylamine, hydroquinone, alpha-tocopherol, tocotrienol, cholecalciferol, propyl gallate, nordihydroquiaretic acid, niacinamide, arginine, Garcinia mangostana (Mangosteen) Peel Extract, Camellia sinensis (Green and White Tea) Leaf Extract, Punica granatum (Pomegranate) Extract, and derivatives, combinations, and mixtures thereof.

9. A topical formulation according to claim 6, wherein said at least one emulsifier/wetting agent is selected from the group consisting of mono and diglycerides and blends thereof, sorbitan fatty acid esters and blends thereof, fatty esters, fatty alcohols, mineral oils, polyether siloxane copolymers, polypropylene glycol (“PPG”)-15 stearyl ether, PPG-10 acetyl ether, PPG-4 lauryl ether, vitamin E acetate, PEG-7 glyceryl, polyoxyethylene sorbitan fatty acid ethers and blends thereof, polyoxyethylene sorbitol esthers, polyoxyethylene acids and blends thereof, polyoxyethylene acids and blends, polyoxyethylene alcohols and blends, polyoxyethylene adducts, ionic surfactants, calcium stearoyl lactylate, ceteareth-20, cetearyl glucoside, ceteth-10, ceteth-2, ceteth-20, cholesterol, cocamide MEA, glyceryl laurate, glyceryl stearate, glyceryl stearate and PEG-100 stearate, glyceryl stearate SE, glycol distearate, glycol stearate, isosteareth, lauramide DEA, laureth-23, laureth-4, linoleamide DEA, methyl glucose sesquistearate, oleth-8, oleth-10, oleth-10/polyoxyl 10 oleyl ether NF, oleth-2, oleth-20, PEG-100 stearate, PEG-20 almond glycerides, PEG-20 methyl glucose sesquistearate, PEG-25 hydrogenated castor oil, PEG-30 dipolyhydroxystearate, PEG-4 dilaurate, PEG-40 sorbitan peroleate, PEG-60 almond glycerides, PEG-8 laurate, PEG-80 sorbitan laurate, polysorbate 20, polysorbate 60, polysorbate 80, polysorbate 85, sodium stearoyl lactylate, sorbitan isostearate, sorbitan laurate, sorbitan oleate, sorbitan sesquioleate, sorbitan stearate, sorbitan stearate (and) sucrose cocoate, sorbitan trioleate, stearamide MEA, steareth-2, steareth-21 and derivatives, combinations and mixtures thereof.

10. A topical formulation according to claim 1, wherein said at least one cannabinoid compound, said at least one skin protecting/enhancing ingredient, and said pharmaceutical carrier are combined to form a cream, gel, liquid, lotion, solution, spray, emulsion or a combination thereof.

11. A topical formulation according to claim 1, wherein said at least cannabinoid compound is about 0.01% to about 25% of said topical formulation.

12. A topical formulation according to claim 1, wherein said at least one cannabinoid compound is about 0.05% to about 4%, about 0.1% to about 6%, about 0.4% to about 8%, about 0.5% to about 10%, about 1% to about 12%, about 1.1% to about 14%, about 1.5% to about 15%, about 2% to about 18%, about 2.5% to about 20%, about 3% to about 22%, about 3.5% to about 24%, about 4% to about 25%, or more of the topical formulation.

13. A topical formulation according to claim 1, wherein said at least one skin protecting/enhancing agent is about 0.01% to about 50% of said topical formulation.

14. A topical formulation according to claim 1, wherein said at least skin protecting/enhancing agent is about 1.0% to about 50%, about 2% to about 18%, about 2.5% to about 20%, about 3% to about 22%, about 3.5% to about 24%, about 4% to about 25%, about 5% to about 26%, about 6% to about 27%, about 7% to about 28%, about 8% to about 29%, about 9% to about 30%, about 10% to about 31%, about 11% to about 32%, about 12% to about 33%, abut 13% to about 34%, about 14% to about 35%, about 15% to about 36%, about 16% to about 37%, about 17% to about 38%, about 18% to about 39%, about 19% to about 40%, about 20% to about 41%, about 21% to about 42%, about 22% to about 43%, about 23% to about 44%, about 24% to about 45%, about 25% to about 46%, about 26% to about 47%, about 27% to about 48%, about 28% to about 49%, or about 29% to about 50% of said topical formulation.

15. A topical formulation according to claim 6, wherein said antioxidant is about 0.01% to about 20% of said topical formulation.

16. A topical formulation according to claim 6, wherein said antioxidant is 0<% to about 20%, 0<% to about 5%, about 1% to about 5%, about 2% to about 5%, about 3% to about 5%, about 4% to about 5%, about 5% to about 9%, about 5% to about 8%, about 5% to about 7%, about 5% to about 6%, about 3% to about 4%, about 3% to about 10%, about 3% to about 11%, about 3% to about 12%, about 3% to about 13%, about 3% to about 14%, about 3% to about 15%, about 3% to about 16%, about 3% to about 17%, about 3% to about 18%, about 3% to about 19%, about 3% to about 20%, or about 5% to about 20% of said topical formulation.

17. A topical formulation according to claim 6, wherein said emulsifier/wetting solution is about 1% to about 20% of said topical formulation.

18. A topical formulation according to claim 6, wherein said emulsifier/wetting solution is about 1% to about 20%, about 5% to about 20%, about 5% to about 15%, about 6% to about 14%, about 7% to about 13%, about 7% to about 12%, about 7% to about 11%, about 7% to about 10%, about 7% to about 9%, about 7% to about 8%, about 10% to about 13%, about 10% to about 12%, about 10% to about 11% or about 9% to about 11% of said topical formulation.

19. A topical formulation according to claim 1, wherein said aqueous component is about 35% to about 99% of said topical formulation.

20. A topical formulation according to claim 1, wherein said aqueous component is about 50% to about 95%, about 55% to about 95%, about 58% to about 95%, about 60% to about 95%, about 65% to about 95%, about 70% to about 95%, about 75% to about 95%, about 80% to about 95%, about 55% to about 60%, about 55% to about 65%, about 55% to about 70%, about 55% to about 75%, about 55% to about 80%, about 55% to about 85%, or about 65% to about 90% of said topical formulation.

21. A topical formulation according to claim 1, wherein said at least one cannabinoid compound is present in an amount therapeutically effective to improve the symptoms and side effects of disorders, diseases and their relative treatments' side effects and symptoms.

22. A topical formulation according to claim 1, wherein said at least one skin protecting/enhancing agent is present in an amount effective to prevent, treat skin conditions and/or enhance the appearance of skin.

23. A topical formulation according to claim 21, wherein said skin conditions is selected from the group consisting of external skin damage, skin damage resulting from reactive oxygen species, chemicals, drug therapies, radiation, skin irritants, inflammation, cutaneous skin aging, rashes, chapping, chaffing, bites, burns, cuts, and scrapes.

24. A topical formulation, comprising: (a) about 0.01% to about 25% of a therapeutically effective amount of at least one cannabinoid compound; and (e) about 0.01% to about 50% of at least one skin protecting/enhancing ingredient; and (c) a pharmaceutical carrier that does not require phospholipids or harsh irritating penetration enhancers effective for simultaneous transdermal delivery of said at least one cannabinoid compound and topical delivery of said skin protecting/enhancing ingredient.

25. A topical formulation according to claim 24, wherein said pharmaceutical carrier includes a wetting agent/emulsifier and an antioxidant and an aqueous solution.
